(19) United States
(12) Patent Application Publication
(10) Pub. No.: US 2018/0369308 A1
(43) Pub. Date: Dec. 27, 2018

(54) CHINESE HERBAL MEDICINE COMPOSITION HAVING A FUNCTION OF RELIEVING HYPOTHYROIDISM, PREPARATION METHOD AND USE THEREOF

(71) Applicant: INFINITUS (CHINA) COMPANY LTD., Jiang Men (CN)

(72) Inventors: Lingyun XIAO, Jiang Men (CN); Renhuai CONG, Jiang Men (CN); Minghua HU, Jiang Men (CN); Yuanyuan WANG, Jiang Men (CN); Fangli MA, Jiang Men (CN); Chung Wah MA, Jiang Men (CN)

(73) Assignee: INFINITUS (CHINA) COMPANY LTD., Jiang Men (CN)

(21) Appl. No.: 16/015,660

(22) Filed: Jun. 22, 2018

(30) Foreign Application Priority Data
Jun. 26, 2017 (CN) 201710494890.X

(51) Int. Cl. A61K 36/258; A61K 36/815; A61K 36/54; A61P 5/14

(52) U.S. Cl. CPC A61K 36/258; A61K 36/815; A61K 2236/331; A61P 5/14; A61K 36/54

(57) ABSTRACT

The present disclosure relates to the field of health care product and food technology, disclosing a Chinese herbal medicine composition having a function of relieving hypothyroidism, and preparation method and use thereof. The Chinese herbal medicine composition described in the present disclosure, in parts by weight, comprises: 10 to 100 parts of GINSENG RADIX ET RHIZOMA, 10 to 100 parts of LYCII FRUCTUS, and 6 to 60 parts of CINNAMOMI CORTEX. The Chinese herbal medicine composition described in the present disclosure adopts GINSENG RADIX ET RHIZOMA, LYCII FRUCTUS and CINNAMOMI CORTEX as starting materials, creates synergistic effects through rational combination. Experiments indicate that the Chinese herbal medicine composition described in the present disclosure can improve functions of the thyroid gland in rat hypothyroidism model, reduce serum VEGF level in the model, showing a function of relieving hypothyroidism and protecting the thyroid gland.

### CROSS REFERENCE TO RELATED APPLICATIONS

This application claims the priority of Chinese Patent Application No. 201710494890.X, filed on Jun. 26, 2017, and the disclosures of which are hereby incorporated by reference.

### FIELD

The present disclosure relates to the field of health care product and food technology, specifically to a Chinese herbal medicine composition having a function of relieving hypothyroidism, and preparation method and use thereof.

### BACKGROUND

Hypothyroidism is a systemic hypometabolic syndrome which is induced by hypothyroidism or thyroid hormone resistance caused by various reasons. Its pathological feature is that mucopolysaccharide accumulates in tissues and skin, manifested as myxedema. According to the site of pathological changes, hypothyroidism is classified into primary hypothyroidism, central hypothyroidism, and thyroid hormone resistance syndrome. Among them, primary hypothyroidism is caused by lesions of the thyroid gland itself; central hypothyroidism is caused by the decrease of thyrotropin-releasing hormone (TRH) or thyroid-stimulating hormone (TSH) production and secretion which is caused by the lesions of hypothalamus and pituitary gland; thyroid hormone resistance syndrome is a syndrome due to a fact that thyroid hormone cannot execute its biological effect in peripheral tissues.

The general clinical manifestations of hypothyroidism are fatigue, cold, weight gain, hypomnesia, diminished response, drowsiness, mental depression, etc., concomitantly with muscle weakness, transient muscle stiffness and other muscle and joint reactions, and when cardiovascular system is involved, it commonly manifests as myocardial myxedema-caused myocardial contractility damage, bradycardia, decreased cardiac output, etc., and anorexia, abdominal distension, constipation and other digestive system reactions often occur. Besides, myxedema coma often occurs in seriously ill patients.

The thyroid gland mainly synthesizes and secretes two types of thyroid hormone having biological activity, one is tetraiodothyronine, i.e. thyroxine (FT4); the other one is triiodothyronine (FT3). Thyroid hormone has a wide range of physiological effects in the body, wherein the most important one is to promote oxidation and heat production in tissues, regulate or promote the development and maturity of human body, functional status of nervous system and cardiovascular system, and metabolism of some substances. Under physiological conditions, most FT3 and FT4 bind to plasma proteins, only a few are free and are called free thyroid hormone (i.e. FT3, FT4). Although the amounts of FT3 and FT4 are small, they can pass through plasma membrane to exert biological effect on target tissue cells, and they are the most sensitive indicators directly reflecting function of the thyroid gland.

At present, oral administration of thyroid tablets or levothyroxine is primarily used in early stage of mild hypothyroidism cases. However, oxygen supply, infusion, infection control, heart failure management and other symptomatic treatments are necessary besides oral administration of thyroid tablets or L-thyroxine in middle and late stages of severe hypothyroidism cases. It is generally a lifelong treatment starting at a low dose and moving up to a maintenance dose while rechecking thyroid function indicators regularly, so the treatment is relatively tedious.

Traditional Chinese medicine treatment has mild side effect so that capture more and more people's attention. Traditional Chinese medicine believes that hypothyroidism belongs to the categories of “consumptive disease”, “edema”, “five retardations”, etc. It is mainly caused by innate deficiency or acquired dystrophy which results in spleen-kidney yang deficiency; or is caused by surgery or medication damages, damaged body yang qi, which results in spleen-kidney yang qi depletion and then disease occurs, and the key to the pathogenesis is spleen-kidney yang deficiency.

### SUMMARY

In view of the above, the purpose of the present disclosure is to provide a Chinese herbal medicine composition capable of relieving hypothyroidism, and preparation method and use thereof.

In order to achieve the purpose of the present disclosure, the present disclosure adopts the following technical solutions:

A Chinese herbal medicine composition, in parts by weight, comprising: 10 to 100 parts of GINSENG RADIX ET RHIZOMA, 10 to 100 parts of LYCII FRUCTUS, and 6 to 60 parts of CINNAMOMI CORTEX.

All the starting materials of the Chinese herbal medicine composition described in the present disclosure are medical and edible dual-purpose plants. Panax ginseng C. A. Mey. is a perennial herb known as the “King of Herbs”. Shennongbencaojing believes that GINSENG RADIX ET RHIZOMA has the efficacy of “five viscera tonification, essence-spirit tranquilize, ethereal soul and corporeal soul tranquilize, fright palpitations relief, pathogen relief, vision improvement, heart open and intelligence reinforcement”. Research indicates that ginsenoside can up-regulate the expression of GATA-3 and Foxp3, down-regulate the expression of T-bet, inhibit hyperactivity of Th1 cells, and regulate immune pathway to exert thyroid protective effect. LYCII FRUCTUS is an important medicinal plant resource in China. It was recorded in the Compendium of Materia Medica that “picking leaves of Lycium barbarum L. in spring, naming it as wolfberry leaves; picking flowers in summer, naming it as Sempervivum; picking seeds in fall, naming it as Lycium barbarum seed; picking roots in winter, naming it as Cortex Lycii”, combining the four flavors, a senior who took it often “lived to or beyond the age of 100 years, walked as if on wings, white hairs turned black again, teeth fell but grew back, made fluid yang and became strong and health”. LYCII FRUCTUS has the efficacies of regulating human body immunity, inhibiting tumor growth, preventing and blocking cancerous cell mutations, etc.; Lycium barbarum polysaccharide has effects of lowering blood lipids levels, lowering blood sugar levels, anti-fatty liver, etc., and also anti fatigue, anti-aging, scavenging free radicals and inhibiting the generation of oxidized lipids. CINNAMOMI CORTEX is the dried bark of Cinnamomum cassia tree of the family Polygonaceae, having the efficacies of “conducting fire back to its origin, warming the kidney and assisting yang, warming the middle and tonifying yang, and dissipating cold and relieving pain”. Widely applied in digestive system, cardiovascular system, immune system and other aspects.

The starting materials of the Chinese herbal medicine composition described in the present disclosure are GINSENG RADIX ET RHIZOMA, LYCII FRUCTUS and CINNAMOMI CORTEX. It creates synergistic effects between medications by using rational combination, and the effectiveness of which is better than that when separately using each one along. In addition, the composition provided by the present disclosure does not include scarce medicines or other medicinal materials that are not food supplements, the effectiveness, however, is better than that of the recipe in which other medications are added.

Furthermore, the experiments of the present disclosure indicate that it is necessary to combine GINSENG RADIX ET RHIZOMA, LYCII FRUCTUS and CINNAMOMI CORTEX within a certain proportion range to create synergistic effects; it will not show excellent effects when the range is outside the proportion range.

Preferably, in parts by weight, the Chinese herbal medicine composition is made from the following starting materials: GINSENG RADIX ET RHIZOMA 20 to 80 parts, LYCII FRUCTUS 20 to 80 parts, and CINNAMOMI CORTEX 12 to 48 parts.

More preferably, in parts by weight, the Chinese herbal medicine composition is made from the following starting materials: GINSENG RADIX ET RHIZOMA 30 to 70 parts, LYCII FRUCTUS 30 to 70 parts, and CINNAMOMI CORTEX 18 to 42 parts.

In a specific embodiment provided by the present disclosure, in parts by weight, the Chinese herbal medicine composition is made from the following starting materials: GINSENG RADIX ET RHIZOMA 10 parts, LYCII FRUCTUS 100 parts, and CINNAMOMI CORTEX 6 parts.

In another specific embodiment provided by the present disclosure, in parts by weight, the Chinese herbal medicine composition is made from the following starting materials: GINSENG RADIX ET RHIZOMA 100 parts, LYCII FRUCTUS 10 parts, and CINNAMOMI CORTEX 60 parts.

In another specific embodiment provided by the present disclosure, in parts by weight, the Chinese herbal medicine composition is made from the following starting materials: GINSENG RADIX ET RHIZOMA 20 parts, LYCII FRUCTUS 80 parts, and CINNAMOMI CORTEX 12 parts.

In another specific embodiment provided by the present disclosure, in parts by weight, the Chinese herbal medicine composition is made from the following starting materials: GINSENG RADIX ET RHIZOMA 80 parts, LYCII FRUCTUS 20 parts, and CINNAMOMI CORTEX 48 parts.

In another specific embodiment provided by the present disclosure, in parts by weight, the Chinese herbal medicine composition is made from the following starting materials: GINSENG RADIX ET RHIZOMA 30 parts, LYCII FRUCTUS 70 parts, and CINNAMOMI CORTEX 18 parts.

In another specific embodiment provided by the present disclosure, in parts by weight, the Chinese herbal medicine composition is made from the following starting materials: GINSENG RADIX ET RHIZOMA 70 parts, LYCII FRUCTUS 30 parts, and CINNAMOMI CORTEX 42 parts.

In another specific embodiment provided by the present disclosure, in parts by weight, the Chinese herbal medicine composition is made from the following starting materials: GINSENG RADIX ET RHIZOMA 50 parts, LYCII FRUCTUS 50 parts, and CINNAMOMI CORTEX 30 parts.

The present disclosure also provides a preparation method of the Chinese herbal medicine composition, mixing GINSENG RADIX ET RHIZOMA, LYCII FRUCTUS and CINNAMOMI CORTEX, extracting by adding water, and collecting the extract after filtration.

Wherein, the weight of the added water is 8 to 12 times the weight of the starting materials.

The temperature of the extraction is 100° C.; the duration of the extraction is 1.5 to 2.5 h.

In some specific embodiments, the weight of water is 10 times the weight of the starting materials.

Preferably, the temperature of the extraction is 100° C.; the duration of the extraction is 1.5 to 2.5 h.

In some embodiments, the temperature of the extraction is 100° C., and duration is 2 h.

Preferably, the number of times of the extraction is 2 to 3 times.

In some embodiments, the number of times of the extraction is 2 times.

Further, the preparation method also comprises a concentration step.

Preferably, the concentration is 8 to 12 folds enrichment.

In some embodiments, the concentration is 10-fold enrichment.

The preparation of the Chinese herbal medicine composition described in the present disclosure is carried out under atmospheric pressure.

Specifically, the procedure of extraction is:

adding water 10 times the weight of the starting materials to GINSENG RADIX ET RHIZOMA, LYCII FRUCTUS and CINNAMOMI CORTEX, extracting at 100° C. for 2 h, filtering and collecting the extract; adding water 10 times the weight of the starting materials to the filter residues, extracting at 100° C. for 2 h, filtering and collecting the extract; combining the two extracts, the Chinese herbal medicine composition is obtained after a 10-fold enrichment.

Research has confirmed that hypothyroidism will cause a decrease of FT3 and FT4 while an increase of TSH in rats. In studies about goiter and thyroid angiogenesis, vascular endothelial growth factor (VEGF) has been considered as an important factor in promoting angiogenesis in the thyroid gland for a long time. In patients with goiter, by paracrine effect, the permeability of micro blood vessels in the thyroid gland can be increased, vascular endothelial cell division and proliferation are promoted, and angiogenesis is induced, it is one of the important mechanisms of goiter formation.

In a specific embodiment, by giving rats 0.2% propylthiouracil (PTU) to establish rat model and then giving the Chinese herbal medicine composition described in the present disclosure, the influence of the Chinese herbal medicine composition described in the present disclosure on the levels of serum free triiodothyronine (FT3), serum free thyroxine (FT4), thyroid stimulating hormone (TSH) and serum vascular endothelial growth factor (VEGF) are detected. The result shows that the Chinese herbal medicine composition described in the present disclosure can significantly increase FT3 and FT4 levels in rat model while significantly lower TSH level in rat hypothyroidism model. Besides, the Chinese herbal medicine composition described in the present disclosure can also improve VEGF level in rat hypothyroidism model, indicating that the Chinese herbal medicine composition described in the present disclosure can significantly improve functions of the thyroid gland, lower serum VEGF level, relieve hypothyroidism.

Therefore, the present disclosure provides a use of the Chinese herbal medicine composition described in the present disclosure for the manufacture of a product for relieving hypothyroidism and protecting the thyroid gland.

The product may be a health care product or food.

According to the demands of different users, the Chinese herbal medicine composition described in the present disclosure can be added to various conventional additives, e.g. disintegrants, lubricants, emulsifiers, adhesives, which are required when preparing different dosage forms, by using conventional formulation methods, to make commonly used medicines or food supplements, e.g. capsule preparations, tablet preparations, pill preparations, oral liquids, powders, soluble granules, powder preparations and so on.

The present disclosure also provides a product for relieving hypothyroidism, comprising the Chinese herbal medicine composition described in the present disclosure.

The product for relieving hypothyroidism described in the present disclosure may be solid preparations, may also be liquid preparations.

Wherein, the solid preparations may be tablet preparations, capsule preparations, soluble granules, pill preparations, dropping pills, mixtures or power preparations.

The liquid preparations may be oral liquid preparations, soft capsule preparations or aerosol preparations.

As can be seen from the technical solution above, the Chinese herbal medicine composition described in the present disclosure adopts GINSENG RADIX ET RHIZOMA, LYCII FRUCTUS and CINNAMOMI CORTEX as starting materials, creates synergistic effects through rational combination. Experiments indicate that the Chinese herbal medicine composition described in the present disclosure can improve functions of the thyroid gland in rat hypothyroidism model, reduce serum VEGF level in the model, showing a function of relieving hypothyroidism and protecting the thyroid gland. In addition, the Chinese herbal medicine composition described in the present disclosure has a simple formula, good safety, easy preparation and clear treatment effect.

### BRIEF DESCRIPTION OF THE DRAWINGS

In order to more clearly illustrate technical solutions of the present disclosure or the conventional technology, the drawings required in description of the embodiments and the conventional technology are briefly described below.

FIGS. 1A, 1B and 1C show statistical graphs of thyroid function indicators in the serum of each group, wherein FIG. 1A is a statistical graph of serum FT3 in each group, FIG. 1B is a statistical graph of serum FT4 in each group, FIG. 1C is a statistical graph of serum TSH in each group; * indicates P<0.05, compared with model group;

FIG. 2 shows a statistical graph of serum VEGF level of each group; * indicates P<0.05, compared with model group.

### DETAILED DESCRIPTION

The present disclosure discloses a Chinese herbal medicine composition having a function of relieving hypothyroidism, and preparation method and use thereof. A person having ordinary skill in the art can learn from the contents herein and improve the process parameters as appropriate. It is specifically to be noted that all similar substitutions and modifications will be apparent to a person having ordinary skill in the art and are to be considered as included in the present disclosure. The method and product of the present disclosure have been described with reference to the preferred embodiments. Apparently, a person having ordinary skill in the art can make modifications and changes to the methods and applications herein without departing from the content, spirit and scope of the present disclosure, to realize and use the present disclosure.

For a further understanding of the present disclosure, the technical solutions in the embodiments of the present disclosure will be clearly and completely described below with the reference to the embodiments of the present disclosure. Obviously, the described embodiments are only a part of, instead of all of, the embodiments of the present disclosure. All other embodiments obtained by a person having ordinary skill in the art based on the embodiments of the present disclosure without creative work shall fall within the scope of protection of the present disclosure.

Unless otherwise specified, all the reagents involved in the embodiments of the present disclosure are commercially available products and can be purchased through commercial channels.

### EXAMPLE 1

### The Chinese Herbal Medicine Composition of the Present Disclosure

The composition was made from the following weight proportion of starting materials: GINSENG RADIX ET RHIZOMA 50 parts, LYCII FRUCTUS 50 parts, and CINNAMOMI CORTEX 30 parts.

The preparation method: 50 parts of GINSENG RADIX ET RHIZOMA, 50 parts of LYCII FRUCTUS, and 30 parts of CINNAMOMI CORTEX was taken, and water 10 times the weight of the starting materials was added. The resulting mixture was boiled and kept boiling for 2 h for extraction. The extraction was performed 2 times. The two filtrates were combined and concentrated until 10-fold enrichment.

### EXAMPLE 2

### The Chinese Herbal Medicine Composition of the Present Disclosure

The composition was made from the following weight proportion of starting materials: 70 parts of GINSENG RADIX ET RHIZOMA, 30 parts of LYCII FRUCTUS, and 42 parts of CINNAMOMI CORTEX.

The preparation method was the same as that of Example 1.

### EXAMPLE 3

### The Chinese Herbal Medicine Composition of the Present Disclosure

The composition was made from the following weight proportion of starting materials: 30 parts of GINSENG RADIX ET RHIZOMA, 70 parts of LYCII FRUCTUS, and 18 parts of CINNAMOMI CORTEX.

The preparation method was the same as that of Example 1.

### EXAMPLE 4

### The Chinese Herbal Medicine Composition of the Present Disclosure

The composition was made from the following weight proportion of starting materials: 100 parts of GINSENG RADIX ET RHIZOMA, 10 parts of LYCII FRUCTUS, and 60 parts of CINNAMOMI CORTEX.

The preparation method was the same as that of Example 1.

### EXAMPLE 5

### The Chinese Herbal Medicine Composition of the Present Disclosure

The composition was made from the following weight proportion of starting materials: 10 parts of GINSENG RADIX ET RHIZOMA, 100 parts of LYCII FRUCTUS, and 6 parts of CINNAMOMI CORTEX.

The preparation method was the same as that of Example 1.

### COMPARATIVE EXAMPLE 1

GINSENG RADIX ET RHIZOMA, the preparation method was the same as that of Example 1.

### COMPARATIVE EXAMPLE 2

LYCII FRUCTUS, the preparation method was the same as that of Example 1.

### COMPARATIVE EXAMPLE 3

CINNAMOMI CORTEX, the preparation method was the same as that of Example 1.

### COMPARATIVE EXAMPLE 4

The composition was made from the following weight proportion of starting materials: 50 parts of GINSENG RADIX ET RHIZOMA and 50 parts of LYCII FRUCTUS.

The preparation method was the same as that of Example 1.

### COMPARATIVE EXAMPLE 5

The composition was made from the following weight proportion of starting materials: 50 parts of GINSENG RADIX ET RHIZOMA and 30 parts of CINNAMOMI CORTEX.

The preparation method was the same as that of Example 1.

### COMPARATIVE EXAMPLE 6

The composition was made from the following weight proportion of starting materials: 50 parts of LYCII FRUCTUS and 30 parts of CINNAMOMI CORTEX.

The preparation method was the same as that of Example 1.

### COMPARATIVE EXAMPLE 7

The composition was made from the following weight proportion of starting materials: 5 parts of GINSENG RADIX ET RHIZOMA, 105 parts of LYCII FRUCTUS, 3 parts of CINNAMOMI CORTEX.

The preparation method was the same as that of Example 1.

### COMPARATIVE EXAMPLE 8

The composition was made from the following weight proportion of starting materials: 105 parts of GINSENG RADIX ET RHIZOMA, 15 parts of LYCII FRUCTUS, 48 parts of CINNAMOMI CORTEX.

The preparation method was the same as that of Example 1.

### TEST EXAMPLE 1

### Pharmacodynamics Study

In order to verify the efficacy of the Chinese herbal medicine composition described in the present disclosure, the extracts of examples 1 to 5 and comparative examples 1 to 8 were subjected to an efficacy verification test, the details were as follows:

### 1. Test Materials

### 1.1 Test Samples

The extracts of examples 1 to 5 and comparative examples 1 to 8.

### 1.2 Experimental Animals

Healthy SD female rats with a body weight from 160 g to 180 g, SPF class.

### 2. Test Method

### 2.1 Model Establishment and Sample Administration

The rats were randomly divided into the following groups: blank control group, model group, model group+Example 1, model group+Example 2, model group+Example 3, model group+Example 4, model group+Example 5, model group+Comparative Example 1, model group+Comparative Example 2, model group+Comparative Example 3, model group+Comparative Example 4, model group+Comparative Example 5, model group+Comparative Example 6, model group+Comparative Example 7, model group+Comparative Example 8. The blank control group was administered with the same amount of normal saline while other groups were administered with 0.2% propylthiouracil (PTU) (0.5 ml/100 g body weight) via gavage to estanblish the model. The gavage was performed once a day for two consecutive weeks in each group. After two weeks, the animals in each group were administrated with the corresponding samples (based on the amount of crude drug, the dosage is 2.5 g/kg/day) via gavage. The gavage was performed once a day for four consecutive weeks in each group. Meanwhile, PTU was added into drinking water for dose maintenance. The model group was given PTU for dose maintenance. The blank control group was given equal amount of distilled water.

### 2.2 Thyroid Function Indicators Detection in Serum

The administration was completed after 6 weeks and blood from the medial canthus of eyes of the rats in each group was collected. The serum was separated and subjected to detection of levels of serum free triiodothyronine (FT3), serum free thyroxine (FT4), and thyroid stimulating hormone (TSH) by radioimmunoassay kits.

### 2.3 Vascular Endothelial Growth Factor

The vascular endothelial growth factor (VEGF) level was detected by ELISA kits.

### 3. Test Results

### 3.1 Serum Thyroid Function Indicators Detection

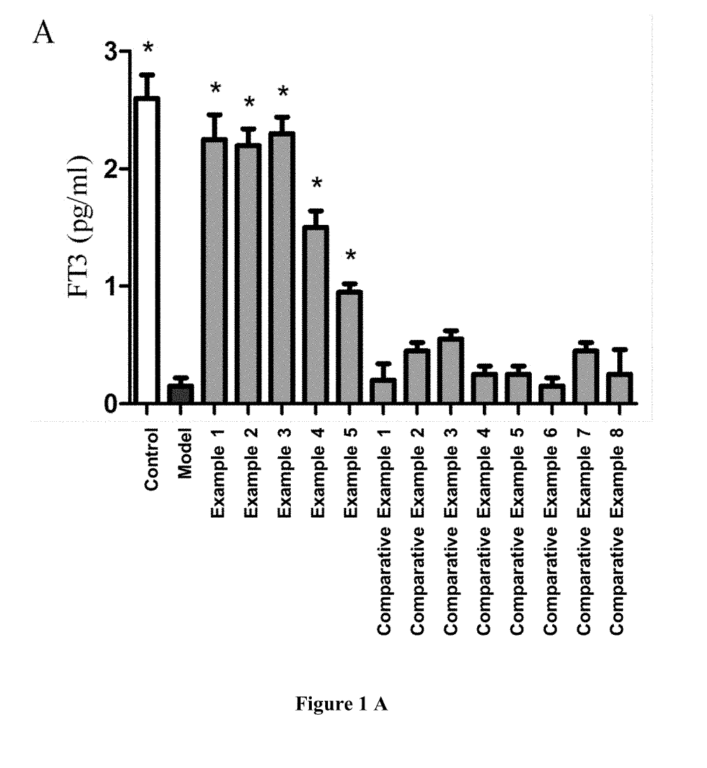

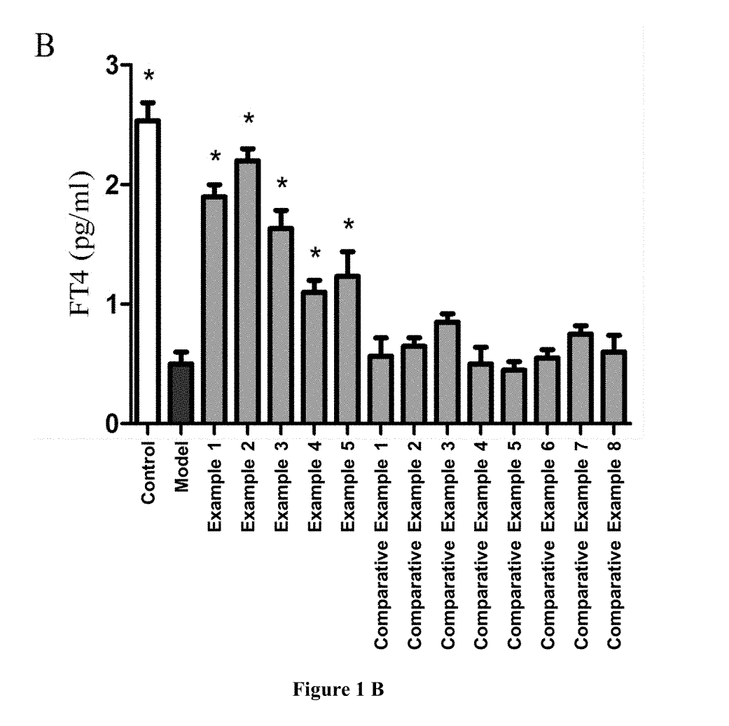

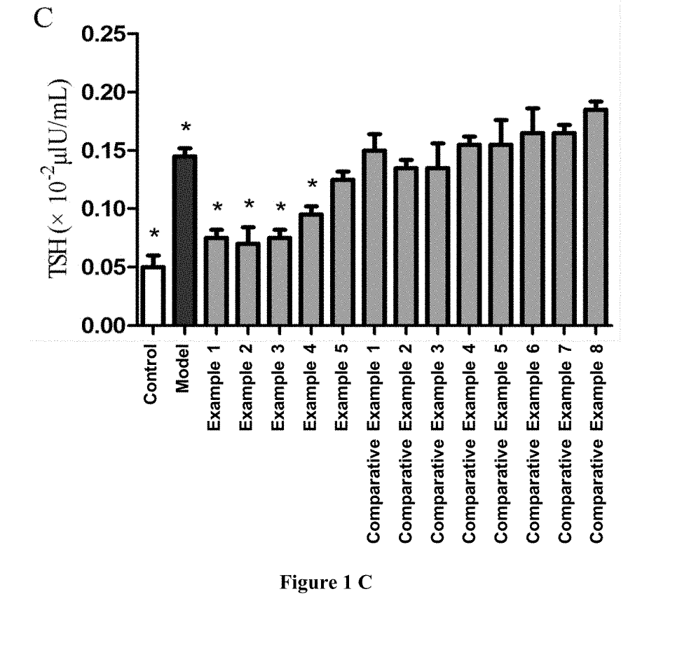

Research report has confirmed that hypothyroidism will cause decreases of FT3 and FT4 while an increase of TSH in rats. The results of the above tests showed that, in the model group, the FT3 and FT4 levels significantly decreased while the TSH level significantly increased, compared with the blank control group. Examples 1 to 5 significantly increased the FT3 and FT4 levels in rat model, examples 1 to 4 significantly lowered the TSH level in rat hypothyroidism model. Comparative examples 1 to 8 did not have significant effects on FT3, FT4 or TSH levels (FIG. 1).

### 3.2 Vascular Endothelial Growth Factor

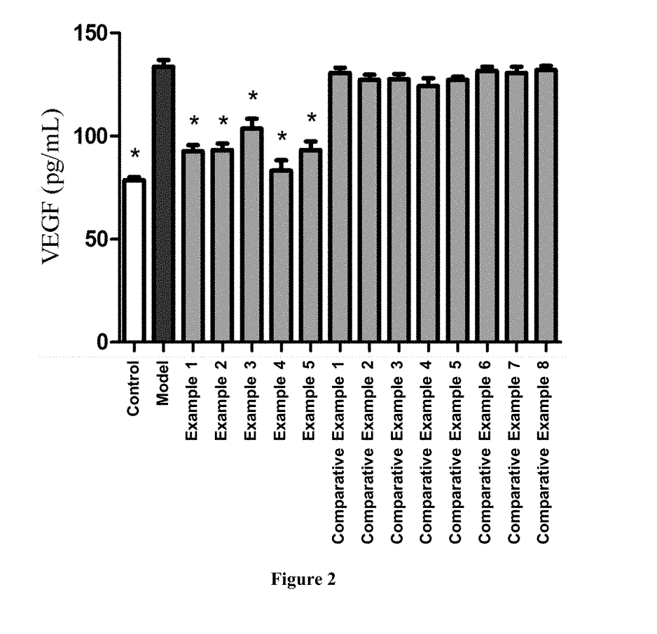

The results of the above tests show that the VEGF level in hypothyroidism model group was significantly higher than that of the blank control group. Examples 1 to 5 significantly improved the VEFG level in rat hypothyroidism model. Comparative examples 1 to 8 did not have significant effects on VEGF level in rat hypothyroidism models (FIG. 2).

To summarize, examples 1 to 5 significantly improved the functions of thyroid gland in rat hypothyroidism model and lowered the serum VEGF level. The compositions of Example 1 to Example 5 have synergistic effects on relieving hypothyroidism.

The above is only preferred embodiments of the present disclosure, and it should be noted that a person having ordinary skill in the art may make some improvements and modifications without departing from the principle of the present disclosure, and these improvements and modifications should also be deemed within the scope of protection of the present disclosure.

What is claimed is:

1. A Chinese herbal medicine composition, in parts by weight, comprising 10 to 100 parts of GINSENG RADIX ET RHIZOMA, 10 to 100 parts of LYCII FRUCTUS, and 6 to 60 parts of CINNAMOMI CORTEX.

2. The composition according to claim 1, wherein, in parts by weight, the composition is made from 20 to 80 parts of GINSENG RADIX ET RHIZOMA, 20 to 80 parts of LYCII FRUCTUS, and 12 to 48 parts of CINNAMOMI CORTEX.

3. The composition according to claim 1, wherein, in parts by weight, the composition is made from 30 to 70 parts of GINSENG RADIX ET RHIZOMA, 30 to 70 parts of LYCII FRUCTUS, and 18 to 42 parts of CINNAMOMI CORTEX.

4. A method for preparing the composition according to claim 1, comprising:
mixing GINSENG RADIX ET RHIZOMA, LYCII FRUCTUS and CINNAMOMI CORTEX as starting materials,
extracting by adding water, and
filtering and collecting the extract.

5. The method according to claim 4, wherein the weight of the added water is 8 to 12 times the weight of the starting materials.

6. The method according to claim 4, wherein the temperature of the extraction is 100° C., the duration of the extraction is 1.5 h to 2.5 h, the times of the extraction is 2 to 3.

7. The method according to claim 4, further comprising a concentration step, wherein the concentration is conducted by concentrating the extract 8 to 12 folds.

8. A method for relieving hypothyroidism and/or protecting functions of the thyroid gland, comprising administering the composition according to claim 1 to a subject in need thereof.

9. The composition according to claim 1, which is in the form of a health care product.

10. The composition according to claim 9, which is in the form of a solid preparation or a liquid preparation; wherein
the solid preparation is a tablet, a capsule, a granule, a pill, a dropping pill, a mixture or a power preparation; and
the liquid preparation is an oral liquid, a soft capsule or an aerosol preparation.
